SEQUENCE LISTING (1 sequence)

SEQ ID NO: 1 - protein, 16 aa, synthetic construct
  source 1..16: note = Description of Artificial Sequence: Synthetic Peptide
RADARADARADARADA

(19) United States
(12) Patent Application Publication
(10) Pub. No.: US 2026/0273018 A1
(43) Pub. Date: Sep. 17, 2026

(54) PHARMACEUTICAL COMPOSITION CONTAINING SPECIFIC SELF-ASSEMBLING PEPTIDE RADA16 FOR TREATING OR PREVENTING LOWER GASTROINTESTINAL DISEASES SUCH AS INFLAMMATORY BOWEL DISEASE

(71) Applicants: 3-D Matrix, Ltd., Newton, MA (US); Gunma University, Gunma Prefecture (JP)

(72) Inventors: Toshio Uraoka, Maebashi City (JP); Daisuke Uehara, Maebashi City (JP); Satoru Kobayashi, Tokyo (JP); Yoshitaka Kanemitsu, Tokyo (JP)

(73) Assignees: 3-D Matrix, Ltd., Tokyo (JP); Gunma University, Gunma Prefecture (JP)

(21) Appl. No.: 19/697,472

(22) Filed: Jun. 3, 2026

Related U.S. Application Data
(62) Division of application No. 18/264,425, filed on Feb. 10, 2022.
(60) Provisional application No. 63/147,945, filed on Feb. 10, 2021.

(51) Int. Cl. A61K 38/10; A61K 9/00; A61K 45/06; A61K 47/36; A61P 1/04

(52) U.S. Cl. CPC A61K 38/10; A61K 9/0043; A61K 9/0095; A61K 45/06; A61K 47/36; A61P 1/04

(57) ABSTRACT

An object of the present invention is to provide a pharmaceutical composition for treating or preventing lower gastrointestinal diseases such as inflammatory bowel disease. The present invention provides a pharmaceutical composition containing a specific self-assembling peptide RADA16 for treating or preventing lower gastrointestinal diseases such as inflammatory bowel disease.

### PRIORITY

This application is a divisional of U.S. Nonprovisional application Ser. No. 18/264,425 filed Aug. 6, 2023, which is a US National Stage entry of International Application No. PCT/US22/15945 filed Feb. 10, 2022, which claims priority to U.S. Provisional Application No. 63/147,945 filed Feb. 10, 2021, the entire contents of each of which are incorporated herein by reference in their entirety.

### SEQUENCE LISTING

The instant application contains an XML Sequence Listing which has been submitted electronically and is hereby incorporated by reference in its entirety. The Sequence Listing, created on Jul. 19, 2023, is named 3DM-21-01-IBD-Gunma-US01_SL.xml and is 2,312 bytes in size.

### TECHNICAL FIELD

The present invention relates to a pharmaceutical composition containing a specific self-assembling peptide RADA16 for treating or preventing lower gastrointestinal diseases such as inflammatory bowel disease.

### BACKGROUND ART

Inflammatory bowel diseases include ulcerative colitis and Crohn's disease, both of which are diseases of unknown cause having repeated exacerbation and remission of chronic inflammation in the intestinal tract, and are designated intractable diseases by the Ministry of Health, Labor and Welfare having no curative treatment. Anti-inflammatory agents such as adrenal cortex hormone, salazosulfapyridine, and mesalazine, immunosuppressive agents such as azathioprine, and antibody formulations such as an anti-TNF-α antibody have been administered independently or in combination according to the medical condition, but none of them are therapeutic agents specific to inflammatory bowel diseases. In addition, adrenal cortex hormones have many side-effects, and long-term administration thereof is not recommended, and anti-TNF-α antibodies have risks of combined serious side-effects such as opportunistic infections.

A peptide having an amino acid sequence indicated by SEQ ID NO 1: RADARADARADARADA (hereinafter abbreviated as RADA16) is known to be specific and have self-assembling characteristics. Due to its physical, chemical, and biological properties, it has recently attracted attention as a material that can be used medically.

### PRIOR ART LITERATURE

Patent Literature 1 JP 2016-515113 A (Translation of PCT Application)
Patent Literature 2 WO 2020/071527

### SUMMARY OF INVENTION

### Problem to be Solved by Invention

An object of the present invention is to provide a pharmaceutical composition for treating or preventing inflammatory bowel diseases in the lower gastrointestinal tract.

### Means for Solving Problem

The present inventors have conducted an intensive search for an effective method for treating inflammatory bowel diseases, and surprisingly, have found that a composition containing a specific peptide (RADA16) is effective, accomplishing the present invention.

That is, one embodiment of the present invention is a pharmaceutical composition for treating or preventing lower gastrointestinal diseases such as inflammatory bowel disease, wherein the pharmaceutical composition relates to a pharmaceutical composition containing a therapeutically effective amount of the specific peptide (RADA16).

One embodiment of the present invention is characterized in that the lower gastrointestinal disease is inflammatory bowel disease.

One embodiment of the present invention is characterized in that the inflammatory bowel disease is ulcerative colitis or Crohn's disease.

One embodiment of the present invention is characterized in that the pharmaceutical composition is locally applied to a target lesion using an endoscope or an anoscope.

In one embodiment of the present invention, the pharmaceutical composition is administered in the form of a suppository.

One embodiment of the present invention is characterized in that the pharmaceutical composition contains a specific peptide having an amino acid sequence indicated by SEQ ID NO 1: RADARADARADARADA at a concentration of 0.1% by weight to 10.0% by weight.

One embodiment of the present invention is characterized in that the pharmaceutical composition further contains another drug effective at treating or preventing lower gastrointestinal diseases such as inflammatory bowel disease or is used in combination with another drug effective at treating or preventing lower gastrointestinal diseases such as inflammatory bowel disease.

One embodiment of the present invention is characterized in that the pharmaceutical composition is used in combination with one or more drugs selected from the group consisting of a duodenal ulcer therapeutic agent, an antidiarrheal, an anti-inflammatory agent, an adrenal corticosteroid, an immunoregulator or immunosuppressive agent, an intestinal mucosa protecting agent, and a blood flow promoting agent. Preferably, the duodenal ulcer therapeutic agent is rebamipide or teprenone, and they are marketed as Mucosta (registered trademark) or Selbex (registered trademark).

One embodiment of the present invention is characterized in that the pharmaceutical composition is used in combination with a cell sheet, an intestinal stem cell, a hematopoietic stem cell, a fat stem cell, or a mesenchymal stem cell.

One embodiment of the present invention is characterized in that the pharmaceutical composition is gelled by self-assembly when applied to a lower gastrointestinal disease site such as an inflammatory bowel disease of interest.

One embodiment of the present invention is characterized in that the specific peptide is a peptide containing SEQ ID NO 1: RADARADARADARADA.

Another embodiment of the present invention relates to a treatment method for lower gastrointestinal diseases such as inflammatory bowel disease, wherein the method includes a step of applying a pharmaceutical composition containing a therapeutically effective amount of a specific peptide to a disease site of a patient. Preferably, the method includes a step of simultaneously or continuously applying another drug effective for the treatment or prevention of lower gastrointestinal diseases such as inflammatory bowel disease.

Another embodiment of the present invention relates to the use of a specific peptide for the production of a therapeutic agent or a prophylactic agent for lower gastrointestinal diseases such as inflammatory bowel disease.

Furthermore, another embodiment of the present invention relates to a pharmaceutical composition for the treatment or prevention of lower gastrointestinal diseases such as inflammatory bowel disease, including a duodenal ulcer therapeutic agent used in combination with a peptide containing the amino acid sequence indicated by SEQ ID NO 1.

The invention optionally combined with one or a plurality of the characteristics described above is also included in the scope of the present invention.

### Effect of Invention

According to the present invention, a safe therapeutic agent and a treatment method for inducing remission for lower gastrointestinal diseases such as inflammatory bowel disease, maintaining remission for a long period, and preventing relapse with extremely few side effects. Also, according to the present invention, even in pathological conditions having a lower gastrointestinal disease such as inflammatory bowel disease, a tendency to suppress body weight loss, reduce increases in DAI score, and suppress reduction in the length of the large intestine is obtained, and there is an effect of reducing the progression of pathological conditions.

### BRIEF DESCRIPTION OF DRAWINGS

The FIGURE shows the histological score results in Example 2.

### MODE(S) FOR CARRYING OUT INVENTION

The present invention relates to a pharmaceutical composition for the treatment or prevention of lower gastrointestinal diseases. The present invention is developed for the treatment of inflammatory bowel diseases, in particular, ulcerative colitis and inflammatory bowel diseases caused by Crohn's disease.

The pharmaceutical composition of the present invention may be used for the treatment or prevention of inflammatory bowel disease of various conditions, and may also be suitably used for the treatment or prevention of intractable inflammatory bowel diseases. In the specification of the present application, “intractable” inflammatory bowel diseases may mean, for example, a disease exhibiting resistance to general preservative medical treatment (for example, nutritional therapy, drug therapy, and the like) for inflammatory bowel diseases.

The subject of application of the present invention may be human or non-human. The non-human subject may be, for example, a non-human animal, and may be, for example, a non-human mammal, bird, reptile, amphibian, or fish. Examples of the non-human mammal include rodents (for example, mice and rats), dogs, cats, horses, pigs, cows, sheep, goats, primates, and the like.

The lower gastrointestinal tract in which inflammatory bowel diseases occur in the present invention includes the small intestine and the large intestine, and includes the colon, the rectum, and the anus.

The method and the route of administration for the pharmaceutical composition of the present invention are not limited, but particularly, it is preferable that the method and the route of administration be topically administrable to a lesion part. For example, topical administration using an endoscope or an anoscope, administration using a nasojejunal tube or an intestinal tube, administration from a surgical incision site of the intestinal tract, and the like may be used.

In one embodiment of the present invention, the pharmaceutical composition is administered in the form of a suppository.

The dosage and the frequency of administration of the pharmaceutical composition of the present invention may be determined appropriately by a person having ordinary skill in the art (for example, a doctor) according to a target pathological condition.

The concentration of the specific peptide in the pharmaceutical composition of the present invention may be 0.1% by weight to 10.0% by weight, and may preferably be 0.3% by weight to 8.0% by weight, more preferably 0.5% by weight to 5.0% by weight, and most preferably 1.0% by weight to 3.0% by weight.

The pharmaceutical composition of the present invention may further contain another drug effective at treating or preventing lower gastrointestinal diseases such as inflammatory bowel disease, or may be used in combination with another drug effective at treating or preventing lower gastrointestinal diseases such as inflammatory bowel disease. The aspect of combining use of the pharmaceutical composition of the present invention and the other drug is not limited, and for example, may be an aspect in which separately prepared respective drugs are administered simultaneously to the subject, or may be an aspect in which the respective drugs are administered to the subject at different times.

The other drugs which may be used together with the pharmaceutical composition of the present invention may, surprisingly, be used for treatment of duodenal ulcers, being for example, rebamipide or teprenone, which are marketed as Mucosta (registered trademark) or Selbex (registered trademark), but the other drug is not limited thereto.

The other drug which may be used together with the pharmaceutical composition of the present invention may be a drug generally used for the treatment of, for example, lower gastrointestinal diseases such as inflammatory bowel disease (ulcerative colitis and Crohn's disease). Examples of the drugs generally used for the treatment of inflammatory bowel diseases (ulcerative colitis and Crohn's disease) include an antidiarrheal, an anti-inflammatory agent, adrenal corticosteroid, an immunoregulator or immunosuppressive agent, an intestinal mucosa protecting agent, and a blood flow promoting agent.

Examples of the antidiarrheal which may be used together with the pharmaceutical composition of the present invention may include diphenoxylate, loperamide, deodorized laudanum, and codeine. Examples of the anti-inflammatory agent which may be used together with the pharmaceutical composition of the present invention may include salazosulfapyridine and an associated drug thereof (mesalazine, olsalazine, balsalazide, and the like). Examples of the adrenal corticosteroid which may be used together with the pharmaceutical composition of the present invention may include prednisolone, budesonide, and hydrocortisone. Examples of the immunoregulator or immunosuppressive agent which may be used together with the pharmaceutical composition of the present invention may include tacrolimus, azathioprine, mercaptopurine, cyclosporine, infliximab, adalimumab, and golimumab.

When the pharmaceutical composition of the present invention is used together with another drug, the ratio of the pharmaceutical composition of the present invention to the other drug may be arbitrary.

The other drugs which may be used together with the pharmaceutical composition of the present invention may be, for example, a composition containing a cell sheet, an intestinal stem cell, a hematopoietic stem cell, a fat stem cell, or a mesenchymal stem cell. The development of mucosal regeneration treatment methods in patients having inflammatory bowel diseases utilizing a cell sheet, a stem cell, and the like, is progressing, and a person having ordinary skill in the art of the present field may use both the treatment via the pharmaceutical composition of the present invention and a mucosal regeneration treatment method utilizing a cell sheet, a stem cell, or the like, in combination. In addition, a synergistic effect may be also expected by mixing the pharmaceutical composition of the present invention with a cell sheet, a stem cell, or the like and applying the mixture to the subject.

The specific peptide RADA16 used in the present invention may be modified (or labeled) as long as main properties of the peptide intended by the present invention are not lost, and peptides modified (or labeled) in this manner are also included in the “specific peptide” in the present invention. The method for modifying (or labeling) the specific peptide used in the present invention may be arbitrary selected by a person having ordinary skill in the art, and may be, for example, an addition of a functional group or the like, an addition of a chemical substance, or addition of an additional protein or peptide. Examples of the addition of the functional group or the like may include acylation, acetylation, alkylation, amidation, biotinylation, formylation, carboxylation, glutamylation, glycosylation (addition of a sugar chain), glycylation, hydroxylation, isoprenylation, lipoylation, addition of a nucleotide or a derivative thereof, polyethylene glycol (PEG)ylation, and addition of a lipid chain. In addition, examples of the addition of the chemical substance may include addition of a suitable labeling agent such as a radioisotope (for example, ¹²⁵I, ¹³¹I, ³H, ¹⁴C, and the like), an enzyme (for example, β-galactosidase, β-glucosidase, alkaline phosphatase, peroxidase, malic acid dehydrogenase, and the like), a fluorescent substance (for example, fluorescamin, fluorescein isothiocyanate, and the like), a luminescent substance (for example, luminol, a luminol derivative, luciferin, lucigenin, and the like), an affinity tag (for example, biotin and the like), In addition, further examples of the addition of the protein or peptide may include ISGylation, SUMOylation, and ubiquitination.

It should be noted that the terms used in the present specification are used to describe a specific embodiment and are not intended to limit the invention.

In addition, the term “include” used in the present specification is intended to indicate the existence of the described matters (such as members, steps, elements, numbers, and the like), except in the case where the context clearly requires a different understanding, and does not exclude the existence of other matters (such as members, steps, elements, numbers, and the like).

Unless defined differently, all terms used herein (including technical and scientific terms) have the same meaning as widely understood by persons having ordinary skill in the art to which the present invention belongs. The terms used herein shall be interpreted as having a meaning consistent with the meaning in the present specification and the relevant technical field, unless a different definition is explicitly stated, and should not be idealized or interpreted in an overly formal sense.

Although terms such as first and second may be used to express various elements, it is understood that these elements should not be limited by those terms. These terms are used only to distinguish one element from other elements, for example, it is possible to describe the first element as the second element, and similarly to describe the second element as the first element without deviating from the scope of the present invention.

The present invention is more specifically described in following examples, but the present invention may be embodied in various forms and should not be interpreted as being limited to examples described here.

### EXAMPLE 1

### A Transanal Administration Experiment on Inflammatory Bowel Disease-Induced Mice

A mouse was made to drink an aqueous solution of 2.5 w/v % dextran sulfate sodium, and the efficacy of a specific peptide having the amino acid sequence indicated by SEQ ID NO 1 on the induced ulcerative colitis model is retrieved by Disease Activity Index. Similarly, Mucosta (registered trademark) and Selbex (registered trademark), which are therapeutic agents for duodenal ulcers, were administered alone or in combination with a peptide having the amino acid sequence indicated by SEQ ID NO 1, and Pentasa (registered trademark) and Rectabul (registered trademark), which are therapeutic agents for ulcerative colitis, were administered alone to compare and study the efficacy. Furthermore, the plasma and removed large intestine collected on the final day of the experiment were shipped to a test contractor.
Dextran sulfate sodium aqueous solution drink period (preparation of dextran sulfate sodium-induced ulcerative colitis model):
   Nov. 16, 2019 to Nov. 25, 2019
Administration period: Nov. 22, 2019 to Nov. 24, 2019
Disease Activity Index (DAI: Evaluation of body weight, properties, and occult blood):
   November 16, November 22, and Nov. 25, 2019
Blood collected: Nov. 25, 2019
Large intestine removed and length measured:
   Nov. 25, 2019
Experiment end date: Nov. 25, 2019

### 1. MATERIALS AND METHODS

### 1.1 Test Materials

Name: Specific peptide having the amino acid sequence indicated by SEQ ID NO: 1 (hereinafter abbreviated as RADA)
   Peptide+Mucosta (hereinafter abbreviated as RADA/Mu)
   Peptide+Selbex (hereinafter abbreviated as RADA/Se)
Provider: Test contractor
   Obtained the prepared liquid.
Properties: Liquid
Storage conditions: Light-shielded, airtight, refrigerated (allowable range: 1.0 to 8.0° C.)
Storage location: Cold storage in the test substance storage room
Treatment of residual portion: Discarded.

### 1.2 Control Substance

### 1.2.1 Negative Control Substance

Name: Japanese pharmacopeia saline solution (hereinafter abbreviated as saline solution)
Manufacturer: Otsuka Pharmaceutical Factory, Inc.
Lot No.: K9D85
Storage conditions: Room temperature (allowable range: 1.0 to 30.0° C.)
Storage location: Test substance storage room

### 1.2.2 Comparative Control Substance

Name: 1) Mucosta (registered trademark) eye drops UD2% (hereinafter abbreviated as Mucosta or Mu)
   2) Selbex (registered trademark) fine grain 10% (hereinafter abbreviated as Selbex or Se)
   3) Pentasa (registered trademark) intestinal infusion 1 g (hereinafter abbreviated as Pentasa or Pen
   4) Rectabul (registered trademark) 2 g intestinal infusion form 14 times (hereinafter, abbreviated as Rectabul or Rec)
Manufacturer: 1) Otsuka Pharmaceutical Co., Ltd.
   2) Eisai Co., Ltd.
   3) Kyorin Pharmaceutical Co., Ltd.
   4) Kissei Pharmaceutical Co., Ltd.
Lot No.: 1), 2) None
   3) R11655A
   4) K037A
Properties: Liquid
Storage conditions: 1), 2) Light-shielded, airtight, refrigerated (allowable range: 1.0 to 8.0° C.)
   3), 4) Light-shielded, airtight, room temperature (allowable range: 1.0 to 30.0° C.)
Storage location: 1), 2) Cold storage in the test substance storage room
   3), 4) Test substance storage room
Treatment of residual portion: Discarded.

### 1.3. Ulcerative Colitis-Inducing Substance

Name: Dextran sulfate sodium 5000 (hereinafter abbreviated as DSS)
Manufacturer: FUJIFILM Wako Pure Chemical Corporation
Lot No.: PTF0313
Storage conditions: Room temperature (allowable range: 1.0 to 30.0° C.)
Storage location: Test substance storage room
Treatment of residual portion: Excluded from the test.

### 1.4 Administration Solution

### 1.4.1 Test Substance, Comparative Control Substance and Negative Control Substance Administration Solution

Preparation method: Since the preparation was used as-is, no preparation was performed.
Treatment of residual fluid after administration: Discarded in an industrial waste container customized for incineration.

### 1.4.2 DSS Aqueous Solution (for Drinking)

Preparation method: 1) Weighed 125.00 g of DSS and transferred to a glass beaker.
   2) Added 4000 mL (80% of the final amount of prepared solution) of water for a water supply used as drinking water for animals in a breeding room, and stirred and dissolved the mixture using a magnetic stirrer.
   3) After dissolution was confirmed, the solution was transferred to a graduated cylinder, the water for the water supply was added, and the solution was measured up to 5000 mL (DSS 2.5 w/v % aqueous solution).
   4) Stirred using a magnetic stirrer.
Storage conditions: Room temperature (allowable range: 1.0 to 30.0° C.)
   The solution was used within four days after preparation, having an expiration date of seven days after preparation.
Storage location: Test substance preparation room and breeding room
Treatment of residual fluid after administration: Discarded in an industrial waste container customized for incineration.

### 1.5 Test System

Species: Mouse (SPF)
Strain: BALB/cAnNCrlCrlj
Source: Charles River Laboratories Japan (Atsugi Breeding Center)
Reason for selection of the test system: The mouse strain is widely used for toxicity and drug efficacy tests.
Sex and order (purchase) count: Male: 60
Age at purchase: 9 weeks
Quarantine and habituation: Quarantine was carried out for six days from the date of animal purchase. During the quarantine period, the general condition was observed daily and body weight was measured on the 1st day (purchase date), 3rd day, and 7th day (quarantine end date). In addition, the habituation period was from the date of animal purchase to the day before the start of drinking the DSS aqueous solution, the general condition was also observed once a day after the quarantine period, and body weight was measured on a grouping day.
Age at the start of test substance administration: 11 weeks

### 1.6 Breeding Conditions

Temperature: Allowable range: 21.0 to 25.0° C.
Humidity: Allowable range: 40.0 to 70.0%
Ventilation number: 15 to 17 times/hour
Lighting time: 12 hours/day (artificial lighting from 7:00 to 19:00)
Feeding method: Feed was put into a feeder made of stainless steel and ingested freely.
Water supplying method:
Before the start of drinking DSS aqueous solution: Water was freely ingested from a glass water dispenser equipped with a touch drink type tipped pipe.
After the start of drinking DSS aqueous solution:
Non-treatment group: Water was freely ingested from a glass or polycarbonate water dispenser equipped with a touch drink type tipped pipe.
Other than non-treatment group: The DSS 2.5 w/v % aqueous solution was freely ingested from a glass or polycarbonate water dispenser equipped with a touch drink type tipped pipe.
Containment in cage: Animals were individually housed in a polycarbonate cage (17.5W×24.5D×12.5H cm having a floor mat).
Cage replacement frequency: At least once every four days (floor water dispenser was also replaced)
Environmental enrichment: During the breeding period, gnawing wood (a wooden piece) was provided in the cage.

### 1.7 Breeding Materials and Analysis

### 1.7.1 Feed

Name: CRF-1 (Solid)
Source: Oriental Yeast Co., Ltd. (Chiba)

### 1.7.2 Drinking Water

Type: Municipal tap water

### 1.7.3 Wooden Piece for Environmental Enrichment

Name: Wooden piece (about 5×2×2 cm high pressure steam sterilization: 121° C. for 20 minutes)
Source: Chiba Animal Materials Co., Ltd.
Material: Fir
Replacement frequency: At least once a week

### 1.8 Individual Identification

Animal: An identification number (1 to 60) was assigned by an ear punch method on the day of animal purchase.
Cage: Using a cage card, the test number, strain, sex, and identification number were displayed on the day of animal purchase, and the group name, animal number and color^{a)} for each group were additionally displayed at the time of grouping.

a)

| | | | |
|---|---|---|---|
| Non-treatment | White | RADA/Se | Purple |
| group: | Black | group: | Pink |
| Control group: | | Mu group: | |
| RADA group: | Blue | Se group: | Green |
| | | Pen group: | Orange |
| RADA/Mu | Red | Rec group | Light blue |
| group: | | | |

### 1.9 Grouping

Grouping period: Day before the start of drinking DSS aqueous solution
   Selection: No abnormalities were observed in the general condition, body weight, or usage of the glass water dispenser before grouping, and thus all cases were subjected to the grouping.
Grouping method: Using a computer program (Provantis system), 50 animals which were close to an average body weight were classified into groups based on body weight on the grouping day, and the animals were randomly assigned to each group in each class. After assigning, animal numbers were randomly given within the group.

| | Animal | Animal | | Animal | Animal |
|---|---|---|---|---|---|
| Group | count | number | Group | count | number |
| Non-treatment | 5 | KX01M1- | RADA/Se | 5 | KX06M1- |
| group | | KX01M5 | group | | KX06M5 |
| Negative | 5 | KX02M1- | Mu group | 5 | KX07M1- |
| control group | | KX02M5 | | | KX07M5 |
| RADA group | 5 | KX03M1- | Se group | 5 | KX08M1- |
| | | KX03M5 | | | KX08M5 |
| RADA/Mu | 5 | KX05M1- | Pen group | 5 | KX09M1- |
| group | | KX05M5 | | | KX09M5 |
| | | | Rec group | 5 | KX10M1- |
| | | | | | KX10M5 |

Handling of non-test animal:
Residual animals at the time of grouping: For the study in section 1.11.3 “Study on Administration Solution Amount,” 10 animals were used.

### 1.10 Dosage

### Production of DSS-Induced Ulcerative Colitis Model>

| | Administ. | Conc. | Administration |
|---|---|---|---|
| Group | sub. name | (w/v %) | route |
| Non-treatment | Water for water | — | Oral (water dispenser) |
| group | supply | | |
| Other than non- | DSS aqueous | 2.5 | Oral (water dispenser) |
| treatment group | solution | | |

### <Treatment of Test Substance>

| | | Administration | |
|---|---|---|---|
| | Administ. | volume* | Administration |
| Group | sub. name | (mL/head) | route |
| Non-treatment group | — | — | — |
| Negative control | Negative control | 0.7 | Transanal |
| group | substance (saline | | |
| | solution) | | |
| RADA group | RADA | 0.7 | Transanal |
| RADA/Mu group | RADA/Mu | 0.7 | Transanal |
| RADA/Se group | RADA/Se | 0.7 | Transanal |
| Mu group | Mu | 0.7 | Transanal |
| Se group | Se | 0.7 | Transanal |
| Pen group | Pen | 0.7 | Transanal |
| Rec group | Rec | 0.7 | Transanal |

*Based on the results of the study in section 1.11.3 “Study on Administration Solution Amount,” the administration volume was set to 0.7 mL.

### 1.11 Administration

1.11.1 Administration route
Administration route: Transanal
   1.11.2 Reason for selection: The same route as the clinical application route was selected.

### Administration Method

Administration method: Performed under anesthesia by inhalation of Japanese pharmacopeia isoflurane (Mylan Inc., hereinafter abbreviated as isoflurane).
   If there is a clot of feces in the rectum, the animal was massaged, and the feces were removed as much as possible.
   The administration solution was aspirated into a 1 mL polypropylene syringe cylinder (sterilized disposable product), inserted through the anus part using a mouse sonde (about 1 cm from the tip), and slowly administered transanally. During administration and for about 10 seconds after the administration, the mouse sonde and the anus part were pressed by hand so that the administration solution does not leak from the anus part. Then, it was slowly pulled out and liquid that leaked from the anus part was wiped off. It should be noted that mixing was performed by lightly inverting by hand so that the test substance administration solution and the Rectabul administration solution would not have large bubbles as much as possible and so that the other comparative control substance administration solution would not foam up, and then each solution was aspirated into the syringe cylinder.
Amount of administration liquid: 0.7 mL
Reason for selection: The method of administration to mice is common and appropriate.

### 1.11.3 Study on Administration Solution Amount

Animal subjects: Of the 10 animals excluded from the grouping, seven animals were used in the study in ascending order of identification number.
Study method: Under isoflurane inhalation anesthesia, 0.7 mL of ink-colored saline solution (calculated as colon diameter: about 3 mm, length from the end of the ileum to the administration site (rectum): about 10 cm) was administered intrarectally, and it was pressed for about 10 seconds so that it would not leak from the anus part. Then, the abdomen was opened and it was confirmed whether the saline solution filled the colon. The same study was also conducted for 0.5 mL.
   When 0.7 mL was administered, since it was confirmed that the saline solution properly filled the colon, 0.7 mL was determined as the amount of the administration solution for the experiment.
Animal treatment: The three surviving animals excluded from the grouping and not used in the study were euthanized by inhalation of carbon dioxide on the day the test substance administration started.
   After the study was completed, the seven animals used in the study were killed by exsanguination by cutting the postcava and abdominal aorta under isoflurane inhalation anesthesia.

### 1.12 Experiment

By setting the day that drinking of the DSS aqueous solution started as Day 1, and the day after that as Day 2, the following days are indicated.

### 1.12.1 Production of DSS-Induced Ulcerative Colitis Model

Animal subjects: All test animals after grouping (excluding non-treatment group)
Model production period: Day 1-10
Model production method: On the day that drinking of the DSS aqueous started (between 13:00 and 14:00), the water in the glass or polycarbonate water dispenser installed in the cage was changed to a DSS 2.5 w/v % aqueous solution, and the solution was freely ingested.

### 1.12.2 Treatment of Test Substance

Animal subjects: All test animals after grouping (excluding non-treatment group)
Treatment date: Day 7, 8, and 9
Number of treatments: Once a day
Treatment time: Between 13:00 and 16:00
Special notes: When the amount of the administration solution specified in section 1.11.3 “Study on Administration Solution Amount” was administered, it was determined that the total solution amount was administered even if it leaked from the anus part.

### 1.12.3 General Condition

Subjects observed: All cases
Observation period: During experimental period
Observation frequency: Once a day (performed before the operation on the day of the operation)
Observation method: Individual observations were performed from outside the cage, and when any abnormalities were suspected, the animals were taken out of the cage and observed.
Special notes: On Days 1, 7, and 10, stool properties and fecal occult blood were Special notes: observed visually and scores were recorded according to the table below.

| Score | Stool properties | Fecal occult blood |
|---|---|---|
| 0 | Normal | None |
| 1 | Soft stool (mild) | + |
| 2 | Soft stool | + + |
| 3 | Mud-like stool | +++ |
| 4 | Watery stool | Gross Bleeding |

### 1.12.4 Body Weight

Subjects measured: All cases
Measurement period and measurement frequency: Days 1, 4, 5, 7, and 10 (performed before administration on the day of administration)
Equipment used: Electronic balance UX4200H (Shimadzu Corporation)

### 1.12.5 Collection of Plasma Sample

Subject of blood collection: All cases except fatal cases
Blood collection period: Day 10
Blood collection site: Postcava
Blood collection amount: 0.4 mL
Plasma amount: 100 μL or more (50 μL or more×2)
Blood collection method: The abdomen of the animal was opened under anesthesia using isoflurane inhalation, and the blood was collected using a 1 mL polypropylene syringe cylinder containing about 2.5 μL of heparin sodium (10,000 units/10 mL) and a 25-gauge syringe needle (both are sterilized disposable products).
Blood processing: The collected blood was placed in a polypropylene tube, immediately ice-cooled, and centrifuged (1600×g, 10 minutes, 4° C.) within 30 minutes after blood collection to obtain plasma. Plasma was aliquoted into two polypropylene tubes of at least 50 μL and frozen until shipping.
Plasma storage conditions: Frozen (allowable range: −95.0° C. to −65.0° C.)
Location to store plasma: Ultra-low-temperature freezer in the freezer room

### 1.12.6 Large Intestine Removal and Length Measurement

Removal subject: All cases except fatal cases
Removal period: Day 10
Removal method: Animals were killed by exsanguination by cutting the postcava and abdominal aorta under isoflurane inhalation anesthesia after blood collection, and the large intestine was removed (from the end of the ileum to the anus part).
Measuring the length of the large intestine: The length (mm) of the removed large intestine was measured using a caliper, and a photograph of the large intestine was taken. Then, the removed large intestine was fixed with 10 vol % neutral buffered formalin solution.
Length of the large intestine: End part of ileum to just before rectum, end of ileum to the anus part

### 1.12.7 Shipping Samples

Date shipped: Dec. 17, 2019
Shipping method: Plasma samples were labeled with the test number, animal number, and blood collection date and sent to the test contractor in a frozen state (packed with dry ice) along with a detailed sample shipping form, and the large intestine fixed with 10 vol % neutral buffer formalin solution was sent to the test contractor at room temperature.

### 1.12.8 Treatment of Dead Animals

Dead animals: Animal numbers KX03M1 and KX04M5
   The body weight was measured immediately after the discovery, and an autopsy was conducted. In addition, the large intestine was removed and fixed with a 10 vol % neutral buffer formalin solution together with the pinna (individual identification) and shipped to the test contractor.

### 1.13 Statistical Analysis

An average value and standard deviation of each group were calculated for the following items.
   Body weight (measured value and rate of change)

Rate of change(% reported digit number: one digit after the decimal point)=(value at each time point-value of the administration date)/value of the administration date×100
Disease Activity Index (DAI)
   According to the score table below, the DAI score is obtained by adding the scores for the body weight, stool properties, and occult blood on days 1, 7, and 10.

| | Rate of body | Stool | Fecal |
|---|---|---|---|
| Score | weight loss | properties | occult blood |
| 0 | None | Normal | Normal |
| 1 | Less than 1 to | Soft stool (mild) | + |
| | 5% | | |
| 2 | Less than 5 to | Soft stool | + + |
| | 10% | | |
| 3 | Less than 10 to | Mud-like stool | + + + |
| | 20% | | |
| 4 | 20% or more | Watery stool | Gross Bleeding |

Length of the removed large intestine
   In the above three items, the F-test was used to perform the equal dispersibility test between the control group and the other group, and a t-test of Student was performed when the dispersion was equal, and a t-test of Aspin-Welch was performed when the dispersion was not equal.

All tests were considered to be significant fluctuations when a difference was observed at the significance level of 5% on both sides, and 5% and 1% were indicated separately in the table.

### 2. RESULTS AND DISCUSSION

### 2.1 Death

### Table 1 Appendices 1, 2, and 3

In one example of the RADA group (animal number KX03M1), a death was observed as follows: <Animal number KX03M1>

In the general condition observations while alive, irregular breathing was observed from Day 6, a small amount of defecation and piloerection were occasionally observed from Day 8, and the subject was found dead on Day 10. Upon being found dead, a dirty part (black) around the anus was observed in the prone position, but no abnormalities were observed in excreted stool. Body weight measurements showed a 21.6% reduction on Day 7 compared to Day 1. During autopsy, a dirty part (black) on the hair around the anus was observed.

In light of these results, the cause of death in the above-mentioned cases was considered to be the progression of DSS-induced ulcerative colitis, based on the results of the surviving cases described later. Subsequent results are described for surviving cases.

### 2.2 General Condition

In the non-treatment group, no abnormalities were observed during the observation period.

The changes observed in the production animals of the DSS-induced ulcerative colitis model were as follows.

A small amount of defecation was observed in one to three cases in the control group, the RADA group, the RADA/Se group, the Se group, the Pen group, and the Rec group, irregular respiration was observed in one to three cases in the RADA group, the RADA/Mu group, the RADA/Se group, the Mu group, the Se group, the Pen group, and the Rec group, piloerection was observed in one case each in the RADA group and the Rec group, black stool was observed in one case each in the RADA group and the Se group, and perianal hair stains (pale red) and anal bleeding were each observed in one case in the RADA group.

Also, in terms of stool properties in the DAI score, one case in the control group scored 1 “soft stool (mild)” on Day 10, and one case in the RADA group scored 1 “+” on Day 10 for fecal occult blood (visual).

These changes were considered to be due to the progress of DSS-induced ulcerative colitis.

### 2.3 Body Weight

In the non-treatment group, no particular fluctuation was observed during the observation period.

In the control group, body weight loss was observed in Day 7 and later, and both the measured body weight and the rate of change in body weight showed significantly lower values on Days 7 and 10 compared to the non-treatment group. In addition, Day 10 showed a lower value than Day 7, so it was confirmed that DSS-induced ulcerative colitis was also progressing during this period.

In the test substance administration group and the comparative control substance administration group, body weight loss was observed on Day 5 and later, and compared to the control group, no significant difference was observed in either the measured body weight value or the body weight change rate except for the measured body weight value on Day 5 for the Mu group. However, on Day 10 of the RADA group and the Mu group, unlike the control group, no further body weight loss was observed compared to Day 7, and thus it was considered that the progression of DSS-induced ulcerative colitis may have been suppressed. It should be noted that the significant low value in the measured body weight value on Day 5 for the Mu group was considered to be due to earlier progression of DSS-induced ulcerative colitis because it was before administration of Mu.

### 2.4 Disease Activity Index (DAI)

The average DAI scores for each group on Days 7 and 10 are as follows:

| | | Negative | | | |
|---|---|---|---|---|---|
| Group | Non-treatment | control | RADA | RADA/Mu | |
| Day 7 | 0.0 | 2.8 | 2.8 | 2.6 | |
| Day 10 | 0.0 | 3.8 | 2.8 | 3.2 | |
| Group | RADA/Se | Mu | Se | Pen | Rec |
| Day 7 | 3.2 | 3.4 | 3.0 | 2.6 | 2.8 |
| Day 10 | 3.2 | 3.0 | 3.4 | 3.8 | 3.6 |

In the non-treatment group, the score was 0.0 on both Days 7 and 10.

The DAI score on Day 10 for the control group showed an increase compared to Day 7, and it was considered that this was due to further progression of ulcerative colitis caused by drinking the DSS aqueous solution.

On Day 10 for the RADA group, the RADA/Se group, and the Mu group, the DAI score did not increase compared to Day 7, and thus, unlike the control group, it was considered that the progression of ulcerative colitis caused by drinking the DSS aqueous solution might have been suppressed.

Meanwhile, on Day 10 for the RADA/Mu group, the Se group, the Pen group, and the Rec group, the DAI score increased compared to Day 7, as in the control group.

### 2.5 Length of Large Intestine

The average values (mm) of the length of the large intestine for each group in the “end part of the ileum to just before the rectum” and the “end part of the ileum to the anus part” are as follows.

| | Non- | Negative | | RADA/ | |
|---|---|---|---|---|---|
| Group | treatment | control | RADA | Mu | |
| End part of the ileum to | 54.55 | 41.73 | 45.52 | 48.06 | |
| just before the rectum | | | | | |
| End part of the ileum to | 95.30 | 69.36 | 75.71 | 78.86 | |
| the anus part | | | | | |
| Group | RADA/Se | Mu | Se | Pen | Rec |
| End part of the ileum to | 48.05 | 50.78 | 48.82 | 49.13 | 45.47 |
| just before the rectum | | | | | |
| End part of the ileum to | 77.64 | 85.00 | 81.08 | 80.99 | 80.34 |
| the anus part | | | | | |

In the control group, compared to the non-treatment group, the lengths of the large intestine of both the “end part of the ileum to just before the rectum” and the “end part of the ileum to the anus part” showed statistically significant low values.

In the test substance administration group and the comparative control substance administration group, compared to the negative control group, the lengths of the large intestine of the “end part of the ileum to just before the rectum” and the “end part of the ileum to the anus part” in the Mu group and the Pen group and the lengths of the large intestine of the “end part of the ileum to the anus part” in the Rec group each showed a significant high value. Moreover, in other groups, the length of the large intestine showed a tendency of shortening suppression.

The comparative control substances Mucosta, Selbex and Rectabul showed an effect of reducing the progression of DSS-induced ulcerative colitis as clear suppression of shortening of the length of the large intestine was observed.

### 3. CONCLUSION

Under the present experimental conditions, in the negative control group, drinking the DSS aqueous solution showed a body weight loss, an increase in the DAI score, and a shortening of the length of the large intestine compared to the non-treatment animals, and thus it was determined that the DSS-induced ulcerative colitis model was produced appropriately.

In these model animals, it was observed that transanally administering the self-assembling peptide (RADA) for three days tended to suppress body weight loss, reduce increases in the DAI score, and suppress shortening of the length of the large intestine, and thus it was considered that it might have an effect of reducing the progression of DSS-induced ulcerative colitis. In addition, from comparing the lengths of the large intestines, it was considered that this reduction effect could be more effective when used in combination with another drug (Mucosta (registered trademark) and Selbex (registered trademark)), but the reduction effect was higher from single drugs of Mucosta (registered trademark), Selbex (registered trademark), and Rectabul (registered trademark).

It should be noted that other peptides having known self-assembling properties were also tested in parallel, and no efficacy was able to be confirmed.

### Example 2

### Histopathological Evaluation of Colon HE-Stained Specimens for Ulcerative Colitis in Mice

A histopathological evaluation of the colon HE-stained specimens prepared in example 1 was performed.
1. Specimen (slide specimen)
Specimen numbers: 1-18
   A number was assigned to each slide, and a corresponding table having the number displayed separately on the slide was created.
2. Histopathological evaluation
Blind evaluation of a pathology score was carried out in accordance with the scoring below for two pieces of the upper and lower parts of the large intestine for each specimen (36 pieces in total). Each score of (1), (2), (3) is multiplied by (4), and the total of these is defined as a “Histological Colitis Score.”¹⁾ It should be noted that the lower part of the large intestine is located on the frost side, and
the upper part of the large intestine is located to the right thereof.

### Histological Assessment of Inflammatory Bowel Disease

| Characteristics | Determination | |
|---|---|---|
| for determination | Criteria | Criteria |
| (1) Inflammation | 0 | None |
| | 1 | Slightly |
| | 2 | Medium |
| | 3 | Severe |
| (2) Range | 0 | None |
| | 1 | Mucous membrane |
| | 2 | Mucous membrane and |
| | | submucosa |
| | 3 | Transmural |
| (3) Crypt damage | 0 | None |
| | 1 | ⅓ of the base is damaged |
| | 2 | ⅔ of the base is damaged |
| | 3 | Only the surface |
| | | epithelium is intact |
| | 4 | Loss of the whole crypt |
| | | and epithelium |
| (4) Impact ratio | 1 | 1-25% |
| | 2 | 26-50% |
| | 3 | 51-75% |
| | 4 | 76-100% |

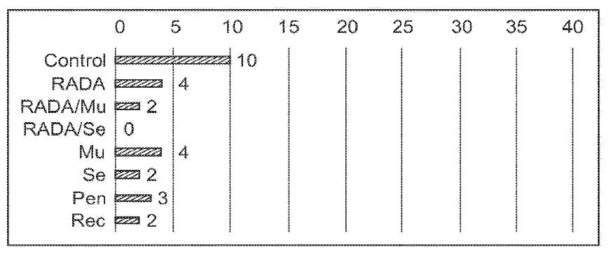

1) Dieleman LA., et al. Chronic experimental colitis induced by dextran sulfate sodium (DSS) is characterized by Th1 and Th2 cytokines. Clin Exp Immunol. 1998. 114:385-391.
The histological score results are shown in FIG. 1.
   From the results, it was found that the administration of RADA alone also had an effect of suppressing intestinal tissue inflammation compared to the negative control, but the combined administration of Mucosta and Selbex was more effective than the administration of each single agent.

1-30. (canceled)

31. A method of treating or preventing a refractory ulcer in the intestinal tract, the method comprising:
a step of applying a pharmaceutical composition containing a therapeutically effective amount of a self-assembling peptide to an ulcer site of the subject having a refractory ulcer in the intestinal tract, wherein the self-assembling peptide is a peptide containing RADARADARADARADA (SEQ ID NO:1), wherein the ulcer in the intestinal tract is caused by ulcerative colitis, Crohn's disease or Behcet's disease,
thereby treating or preventing a refractory ulcer in the intestinal tract.

32. The method according to claim 31, wherein the pharmaceutical composition is applied to the ulcer of a subject having an ulcer in the intestinal tract.

33. The method according to claim 32, wherein the pharmaceutical composition is locally applied to the ulcer using an endoscope or an anoscope.

34. The method according to claim 31, wherein the pharmaceutical composition contains the self-assembling peptide at a concentration of 0.1% by weight to 10.0% by weight.

35. The method according to claim 31, wherein the pharmaceutical composition further contains an additional drug effective in treating or preventing an ulcer in the intestinal tract.

36. The method according to claim 31, wherein the pharmaceutical composition is used in combination with an additional drug effective in treating or preventing an ulcer in the intestinal tract.

37. The method according to claim 35 or 36, wherein the additional drug effective in treating the ulcer in the intestinal tract is one or two or more drugs selected from: an antidiarrheal agent, an anti-inflammatory agent, a corticosteroid, an immunomodulatory agent or immunosuppressive agent, an intestinal mucosal protective agent, and a blood flow accelerating agent.

38. The method according to claim 35 or 36, wherein the additional drug is a composition containing a cell sheet, an intestinal stem cell, a hematopoietic stem cell, an adipose stem cell, or a mesenchymal stem cell.
